(19) United States
(12) Patent Application Publication
(10) Pub. No.: US 2023/0007930 A1
(43) Pub. Date: Jan. 12, 2023

(54) URINE ANALYSIS DEVICES AND METHODS FOR REAL TIME MONITORING OF KIDNEY FUNCTION

(71) Applicant: THE BOARD OF REGENTS OF THE UNIVERSITY OF TEXAS SYSTE, Austin, TX (US)

(72) Inventors: Matthew LEVENO, Dallas, TX (US); Cristhiaan Ochoa ARENAS, Dallas, TX (US); Justin MCFARLANE, McKinney, TX (US); Luis JULE, Grand Prairie, TX (US); Ryan FINNIE, Dallas, TX (US); Saud MUDANI, Wylie, TX (US); Alexander HARPER, McKinney, TX (US); Daniel KAMINSKI, Joshua, TX (US)

(73) Assignee: THE BOARD OF REGENTS OF THE UNIVERSITY OF TEXAS SYSTEM, Austin, TX (US)

(21) Appl. No.: 17/782,402

(22) PCT Filed: Dec. 4, 2020
(86) PCT No.: PCT/US2020/063277

§ 371 Date: Jun. 3, 2022

Related U.S. Application Data
(60) Provisional application No. 62/944,364, filed on Dec. 5, 2019.

(51) Int. Cl. A61B 5/00; A61B 5/145; A61B 5/20; G16H 10/40

(52) U.S. Cl. CPC A61B 5/0022; A61B 5/14507; A61B 5/207; A61B 5/14546; A61B 5/201; G16H 10/40

(57) ABSTRACT

Described herein are devices and methods for continuous real time monitoring of kidney function. In various embodiments, a urine analysis device collects sensor data describing one or more properties of urine. The urine analysis device may be integrated with a catheter system to continuously generate sensor data in real time as the urine is collected by the catheter system. Sensor data collected by the urine analysis device may be analyzed by physicians to detect changes in a patients kidney function. If necessary, based on the sensor data, physicians may perform an intervention to improve a patients kidney function.

### CROSS REFERENCE TO RELATED APPLICATIONS

This application claims the benefit of U.S. Provisional Application Ser. No. 62/944,364 filed Dec. 5, 2019, the entire contents of which is hereby incorporated by reference.

### BACKGROUND

Acute kidney injury (AM) is an abrupt or rapid decline in kidney function. AKI can occur as rapidly as minutes to hours and is an independent predictor of morbidity and mortality. This condition is often caused by inadequate renal perfusion, but may also occur due to direct kidney damage, or blockage within a patient's urinary collection system. It affects more than 200,000 people each year in the United States. AM is not specific to any age group, gender, or ethnicity. However, it typically occurs in people who are already sick or in the ICU.

In traditional clinical settings, two to three variables are assessed to detect acute kidney injury. First, a blood test to measure serum creatinine is performed to estimate the state of kidney function. It is typically measured once per day and concerning trends are often only identifiable after the injury has occurred. The second variable commonly used to monitor kidney function is urine output over time. In many hospitalized patients outside the ICU setting and in the majority of patients in the ICU, urine production may be quantified over time using an indwelling urinary catheter and collection chamber. However, in many patients the urine flow is an insensitive and non-specific indicator of kidney function. A third variable currently used to assess the perfusion state of the kidney is obtained through measurement of urinary electrolytes. Most specifically, urine electrolytes and creatinine can be used to determine how the kidney is handling sodium. Kidneys with an intact response to hypoperfusion avidly reclaim sodium, while those that are either well perfused or damaged will more liberally allow sodium to be excreted. Typically, the urine electrolytes will be measured at a single point in time to better gauge the perfusion state of the kidney. However, it is not common practice to follow these urine electrolytes serially. Should the practice of serial measurement of urine electrolytes be advantageous, there are substantial practical barriers to doing so. As such, devices and methods are needed to monitor changes in urine conductivity as a surrogate for urinary electrolyte content.

### BRIEF DESCRIPTION OF THE FIGURES

FIG. 1 shows a system for urine analysis according to various embodiments of the present disclosure.

FIG. 2 illustrates more details of a urine analysis device integrated with a catheter system according to various embodiments of the present disclosure.

FIG. 3a illustrates more details of a urine analysis device according to various embodiments of the present disclosure

FIG. 3b illustrates a closed configuration of a sensor housing according to various embodiments of the present disclosure.

FIG. 3c illustrates an open configuration of a sensor housing according to various embodiments of the present disclosure.

FIG. 4 illustrates exemplary conductivity sensors integrated with a catheter tube according to various embodiments of the present disclosure.

FIG. 5 is a circuit diagram illustrating exemplary electrical components included in a urine analysis device according to various embodiments of the present disclosure.

FIG. 6 is a flow chart illustrating a method of recording new conductivity measurements according to various embodiments of the present disclosure.

FIG. 7 is a flow chart illustrating a method of using the urine analysis device to monitor kidney function according to various embodiments of the present disclosure.

FIG. 8 is an exemplary conductivity output generated by the urine analysis device according to various embodiments of the present disclosure. Line 808 verifies a trend is present, uploaded via Wi-Fi every 30 seconds, sample rate is 3 seconds. The x axis represents time, a sample was recorded every 30 seconds.

FIG. 9 is a line chart illustrating changes to urine conductivity and sodium concentration over time according to various embodiments of the present disclosure.

FIG. 10 is an exemplary computer system implementing a client device according to various embodiments of the present disclosure.

### SUMMARY

The devices and methods therefore enable a real-time assessment of changes in urinary electrolytes to provide practitioners with a more meaningful and timely signal of compromised and preventable kidney injury.

An embodiment provides a system for urine analysis integrated with a catheter. The system comprises a clamp connected to a catheter tube and a body portion mounted to a urine collection receptacle, the body portion comprising a sensor housing disposing one or more sensors configured to collect sensor data describing one or more properties of urine collected by the catheter; a communications module configured to update a patient record by transmitting the sensor data to a database; and a data analytics module configured to generate one or more data visualizations displaying the sensor data. The one or more sensors can include a conductivity sensor configured to collect conductivity data describing a dissolved ion concentration of urine. The communications module can include a wireless communications module configured to continuously update the patient record in real time by integrating with a cloud database API that interfaces with a patent medical records system. The wireless communications device can be a Wi-Fi chip integrated with a webhook service. The webhook service can be configured to automatically establish a connection with the cloud database API when the wireless communications device receives sensor data. Upon receiving no new sensor data for one or more sampling periods, the communications module can be configured to transition the wireless communications device to a low power state having the Wi-Fi chip powered off. The one or more visualizations can include a graph displaying patient conductivity data collected over a period of time. The data analytics module can be configured to extract one or more trends from the graph by drawing a trendline indicating an increase or decrease in urine conductivity.

Another embodiment provides a urine salt monitoring device integrated with a catheter system. The device comprises a clamp connected to a catheter tube included in the catheter system; a body portion mounted to a urine collection receptacle included in the catheter system; a sensor housing disposed in a cavity included in the body portion, the sensor housing disposing one or more sensors configured to collect sensor data describing one or more properties of urine collected by the catheter; and a processor and memory disposed inside the body portion, the memory including instructions causing the processor to: operate the one or more sensors to generate the sensor data; and continuously upload the sensor data to a database to update a patient record in real time. The one or more sensors can include a conductivity sensor configured to measure the conductivity of urine collected by the catheter system. The conductivity sensor includes two or more electrodes configured to be inserted into the catheter tube to contact urine as it flows through the catheter tube. The device can comprise a cap placed over the sensor housing, the cap having a groove for disposing the catheter tube and one or more openings within the groove. The two or more electrodes can extend through the two or more openings to contact the catheter tube disposed inside the groove. The clamp can be configured to rotate over the groove to compress the catheter tube against the two or more electrodes. The clamp can be dimensioned to force the electrodes to pierce the catheter tube when the clamp is secured over the groove. The device can comprise a top clip and a bottom clip for shaping the catheter tube into an elbow bend that forces a portion of the catheter tube extending over a top surface of the body portion to lay horizontally. The elbow bend can increase dwell time of urine in a portion of the catheter tube disposed inside the clamp.

Yet another embodiment provides a method of using a system for urine analysis integrated with a catheter to determine kidney function. The method comprises measuring conductivity data of urine collected by the catheter; updating a patient record to include the conductivity data; analyzing the patient record to extract one or more trends from the measured conductivity data; and determining kidney function of a patient based on the one or more trends. The one or more trends can comprise a decrease in the patient's urine conductivity over time. The patient's kidney function can be determined to be compromised based on the decrease in the patient's urine conductivity. The method can further comprise converting the measured conductivity data to a urine dissolved salts concentration. The method can further comprise performing an intervention on the patient to improve the patient's kidney function. Interventions can include, for example, restricting the patient's fluid intake, initiating antidiuretic hormone replacement therapy, or administering 3% saline.

### DETAILED DESCRIPTION OF SEVERAL EMBODIMENTS

Disclosed herein are urine analysis devices and methods that enable real time monitoring of kidney function. In various embodiments, the urine analysis devices are integrated with existing catheter systems to expedite adoption in clinical settings. Urine analysis devices may generate real time conductivity data for patient urine. Recorded conductivity data may serve as a proxy for urine salt levels that may be analyzed to extract patterns and/or trends in the function of a patient's kidneys or other organs. Clinicians may interpret these tends to monitor rapid changes in the patient's health. Conductivity data may be continuously generated and recorded to provide a source of real time clinical data that may allow clinicians to make accurate decisions about patient treatment more rapidly than test results return from the lab. Real time urine analysis may also provide a method for fast and precise monitoring of changes in a patient's condition after a treatment is administered. By increasing the amount of data and frequency of new data points, the urine analysis devices and methods described herein may enable more accurate diagnoses and increase the ability of physicians to properly react to changes in a patient's condition.

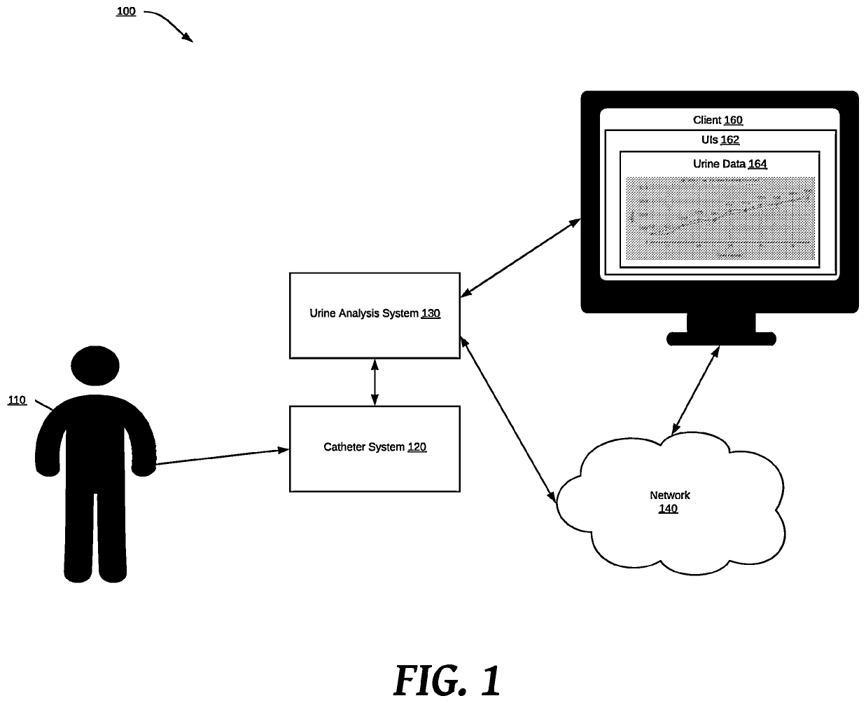

FIG. 1 shows a system for urine analysis according to various embodiments of the present disclosure. System 100 may include a plurality of functional elements that may be provided by mechanical components, electrical components, and or computing devices. These elements may work together to collect urine samples from a patient 110, measure data points describing one or more properties of the urine samples, and analyze the data to detect patterns related to one or more patient conditions.

For example, system 100 may include at least one client 160. Client 160 may be any device configured to present user interfaces (UIs) 162 including one or more records of urine data 164 and receive inputs thereto in the UIs 162. For example, client 160 may be a smartphone, personal computer, tablet, laptop computer, or other device.

System 100 may include a catheter system 120. In some embodiments, the catheter system 120 may a Foley catheter that collects urine from a patent 110. The catheter system 120 may include a tube that is inserted into the patient's bladder to drain urine excreted by the body. The catheter system 120 may also include a receptacle connected to the tube for collecting urine and a mechanism for removing urine from the receptacle.

System 100 may include a urine analysis system 130. In various embodiments, the urine analysis system may be integrated with the catheter system 120 to automatically measure one or more properties of urine collected by the catheter system 120. The urine analysis system may include one or more hardware and/or software components. Exemplary hardware components may include one or more sensors that measure one or more urine properties. Exemplary software components may include modules for analyzing and transmitting sensor data. In some embodiments, the one or more hardware and/or software components may be accessible to the client 160 through a network 140 in some embodiments (e.g., a urine analysis module of the urine analysis system 130 may be hosted by a server computer). As described in greater detail below, the urine analysis system may upload sensor data to the client 160 to enable real time urine analysis.

In some embodiments, one or more clients 160 may communicate with one or more urine analysis systems 130 through a network 140. For example, communication between the elements may be facilitated by one or more application programming interfaces (APIs). APIs of the system 100 may be proprietary and/or may be examples available to those of ordinary skill in the art such as Amazon Web Services (AWS) APIs, Google APIs, and the like. Network 140 may be the Internet and/or other public or private networks or combinations thereof.

A single client 160 and separate, single urine analysis system 130, and/or catheter system 120 are shown for ease of illustration, but those of ordinary skill in the art will appreciate that these elements may be embodied in different forms for different implementations. For example, system 100 may include a plurality of clients 160, many of which may access different data. Moreover, a single urine analysis system 130 be components of a single computing device or a combination of computing devices may provide a single urine analysis system 130. In some embodiments, the operations performed by client 160 and at least one of the separate, single urine analysis systems 130 may be performed on a single device (e.g., without the various components communicating using network 140 and, instead, all being embodied in a single computing device).

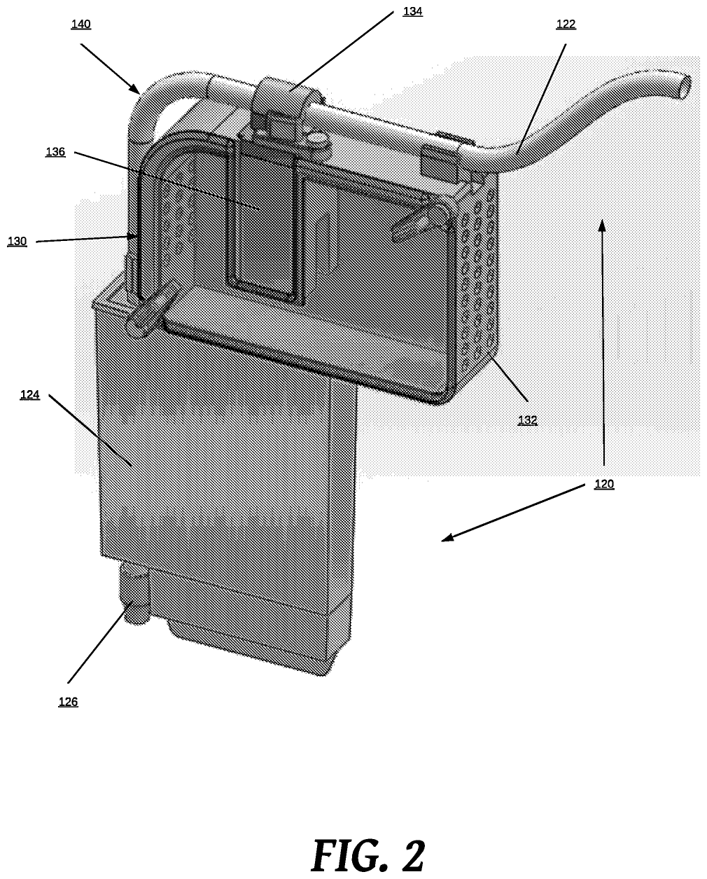

FIG. 2 illustrates an exemplary urine analysis system 130 embodiments integrated with a catheter system 120. As shown, the urine analysis system 130 may attach to a catheter system 120 at various points. In various embodiments, the urine analysis system 130 includes a body portion 132 mounted on a urine collection receptacle 124 and a clamp 134 that secures the catheter tube 122 to the body portion 132. The body portion 132 may be mounted on top of, on the side of, or any other suitable location on the urine collection receptacle 124. The clamp 134 may secure the catheter tube 122 to the top of, the side of, or any other suitable location on the body portion 132. The urine analysis system 130 may be configured to measure properties of the urine as it flows from the patient to the urine collection receptacle 124. In various embodiments, one or more sensors (e.g., 1, 2, 3, 4, 5, or more) inside a sensor housing 136 may pierce the portion of the catheter tube 122 within the clamp 134. The sensor housing 136 may be disposed inside the body portion 132 beneath clamp 134. The catheter system 120 may also include a closable opening 126 on the underside or side of the urine collection receptacle 124 that allows collected urine to be drained from the urine collection receptacle 124. In various embodiments, a urometer that detects the volume of collected by the catheter system 120 may be integrated into the closable opening 126 and/or the urine collection receptacle 124.

To extend the usable life of the urine analysis system 130, one or more portions of the sensor may be modular to enable the portions of the sensor that interface with the urine to be replaced. In various embodiments, the sensor housing 136 may be removed from the body portion to facilitate replacing one or more of the modular portions of the sensor, for example, the sensor probes that pierce the catheter tube and contact the urine. The urine analysis system 130 may also be compatible with one or more types of catheter systems 120. To facilitate adapting the urine analysis system 130 to different catheter systems 120, the clamp 134 and body portion 132 may be removed from one catheter system 120 by removing the catheter tube 122 from the clamp 134 and detaching the body portion 132 from the urine collection receptacle 124. The urine analysis system 130 may then be integrated into a second catheter system by securing the catheter tube 122 of the second catheter system inside the clamp 134 and attaching the body portion 132 to the second system's urine collection receptacle 124.

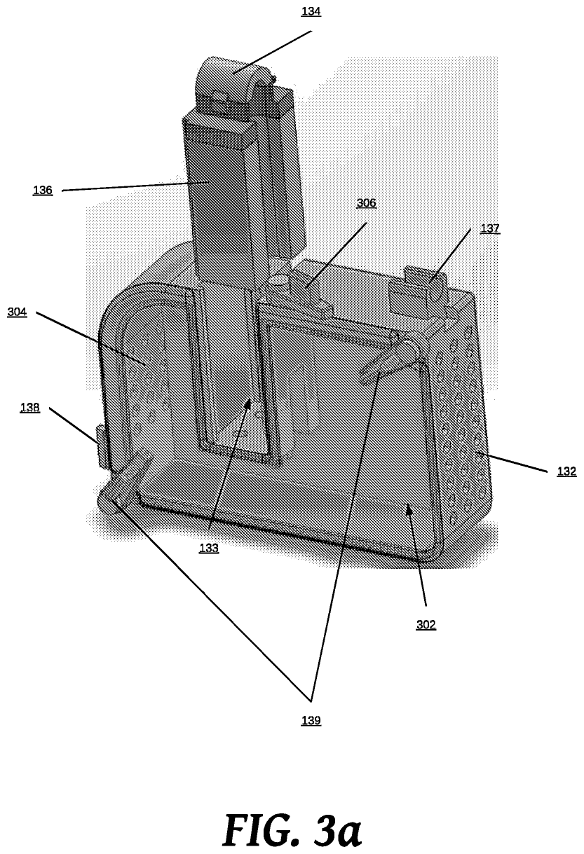

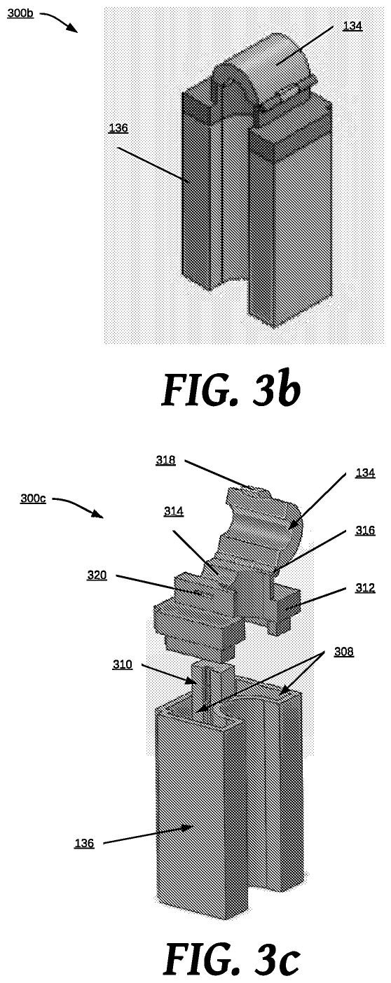

FIG. 3a illustrates an exploded view of the urine analysis system 130. As shown, the body portion 132 includes a sensor cavity 133 for disposing the removable senor housing 136. The exterior walls of the body portion 132 define a main chamber 302 that may be covered with a removable lid 304 (shown as transparent in this embodiment). One or more hinged latches 139 may secure the removable lid 304 to the body portion 132. Electronics components including a printed circuit board including one or more processors, microcontrollers, sensor control circuits, power management circuits, and/or communication modules may be stored inside the main chamber 302 along with a power supply, for example, a battery.

The sensor housing 136 may be secured inside the sensor cavity 133 using one or more sensor latches 306 attached to the main body 132. The sensor latches 306 may be attached to the top of, the side of, or any other suitable location on the main body 132. In various embodiments, the sensor latch 306 and/or the one or more hinged latches 139 include a hinge cap secured over a pin. The hinge cap and pin allow the sensor latch 306 and/or hinge latches 139 to rotate up to 360 degrees. To secure the sensor housing using 136 the sensor latch 306 and/or the removable lid 304 using the one or more hinged latches 139 the latches are rotated over a surface of the sensor housing 136 and/or removable lid 302 to hold these structures in place. The sensor latch 306 and/or one or more hinged latches 139 may then be rotated away from the sensor housing 136 and/or removable lid 302 to allow the structures to be moved.

The sensor housing 136 may include a clamp 134 or other suitable device to hold a catheter tube mounted on top of the housing. The clamp 134 secures a catheter tube to the urine analysis system 130 along with, for example, one or more clips (e.g., 1, 2, 3, 4, or more). As shown, a top clip 137 may clip a top portion of the catheter tube to the top surface of the body portion 132 and a bottom clip 138 may secure a bottom portion of the catheter tube to a side surface of the body portion 132. In various embodiments, the clamp 134, top clip 137, and bottom clip 138 may shape the catheter tube to form an elbow bend 140 including a portion of the catheter tube that lays horizontally flat across the top surface of the body portion 132. The elbow bend 140 formed by the clamp 134 and clips 137, 138 increases the dwell time for urine in the horizontal portion of the catheter tube to give the sensor portions piercing the catheter tube inside the clamp 134 more time to interact with the urine and take measurements. The elbow bend 140 may also improve the accuracy and consistency of measurements taken by the one or more sensors by reducing the flow velocity and stabilizing the amount of liquid present in the catheter tube. The elbow bend 140 may have any angle of bend (e.g., 90°, 60°, 30°, 10°, and the like) suitable to fit the catheter tube around the body portion 132. The angle of bend of the elbow bend 140 may be varied according to the shape of the body portion 132 and the urine dwell time, urine flow velocity, and amount of liquid in the catheter tube required to ensure the one or more sensors can accurately measure conductivity and other properties of the urine. For example, the angle of bend of the elbow bend 140 may be increased (e.g., from 30° to 90°) to reduce the urine flow velocity and increase the urine dwell time and the amount of liquid in the portion of the catheter tube interfacing with the one or more sensors, to improve the accuracy and consistency of measurements taken by the one or more sensors.

FIG. 3b illustrates a sensor housing 136 and clamp 134 in a closed configuration 300b. FIG. 3c illustrates an exploded view of a sensor housing 136 and clamp 134 in an open configuration 300c. As shown in FIG. 3c, the exterior walls of the sensor housing 136 and a central partition 310 may define one or more pockets 308 (e.g., 1, 2, 3, 4, 5, or more) for disposing sensors. In various embodiments, the sensors may be electrodes for sensing one or more properties of urine captured by the catheter system, for example, conductivity, temperature, volume, pH, and the like. The central partition 310 may include one or more channels for guiding an aspect of the electrodes into position, for example, the electrode probes that pierce the catheter tube and contact the urine.

In various embodiments, the space defined by the two pockets 308 may be enclosed by a cap 312 placed over the two pockets 308 and central partition 310. The cap 312 may be removably fixed to the sensor housing 136 so that the housing may be opened to remove and/or replace one or more sensors disposed inside the two pockets 308. The cap 312 may also be permanently fixed to the sensor housing 136. In various embodiments, the clamp 134 is attached to the cap 312 via a hinge mechanism 316 that allows the clamp to be rotated away from and over the top of the cap. The cap may also include a groove 314 cut into the top surface of the cap. To attach the clamp 134 to the catheter tube, the catheter tube may be inserted into the groove 314 and the clamp 134 may be rotated over the catheter tube to secure the tube inside the groove.

In various embodiments, closing the clamp 134 over the catheter tube may generate a force that causes a portion of the electrodes to pierce the catheter tube. To position the probes in contact with the catheter tube, a portion of the electrodes may extend through one or more openings in the groove 314. The channels in the central partition 310 may guide the electrodes through the one or more openings. The channels may also support and secure the electrodes so they don't break, recoil, and/or otherwise displace when pressed against the catheter tube or while the electrodes are contained in the catheter tube. To pierce the catheter tube with the electrodes, the clamp 314 may be rotated over the catheter tube to press the catheter tube against the portion of the electrodes extending through the groove 314. For example, the clamp 134 may compress the catheter tube against the two or more electrodes that extend through the groove 314 when the clamp 134 is closed over the groove 314. The clamp 314 may be secured into place over the catheter tube inside the groove 314 by pressing a tab 318 into a slot 320 included in the cap 312. The clamp 314 may also be secured in place using a clip, pin, latch, snapring, or other suitable mechanical fastener. The size of the opening provided by the groove 314 and the clamp 134 may be configured so that the force required to press the tab 318 into the slot 320 causes the electrodes to pierce the catheter tube and penetrate into the catheter tube so that the electrodes are submerged in the urine or other liquid traveling through the catheter tube. For example, the mechanical force required to close and or secure the clamp 134 over the groove 314 with the catheter tube inside the groove 314 may be sufficient to cause the electrodes to pierce the catheter tube. The size of the clamp 134 may facilitate forcing the electrodes to pierce the catheter tube when the clamp 134 is secured over the groove. For example, the dimensions (i.e., length, width, surface area, and the like) the clamp 134 may exceed the dimensions of the groove 314 so that the clamp 134 compresses a portion of the catheter tube against the complete surface area of the opening of the groove 314. By compressing a portion of the catheter tube over the entire opening of the groove 314, the clamp ensures any electrodes contained within the groove will pierce the catheter tube.

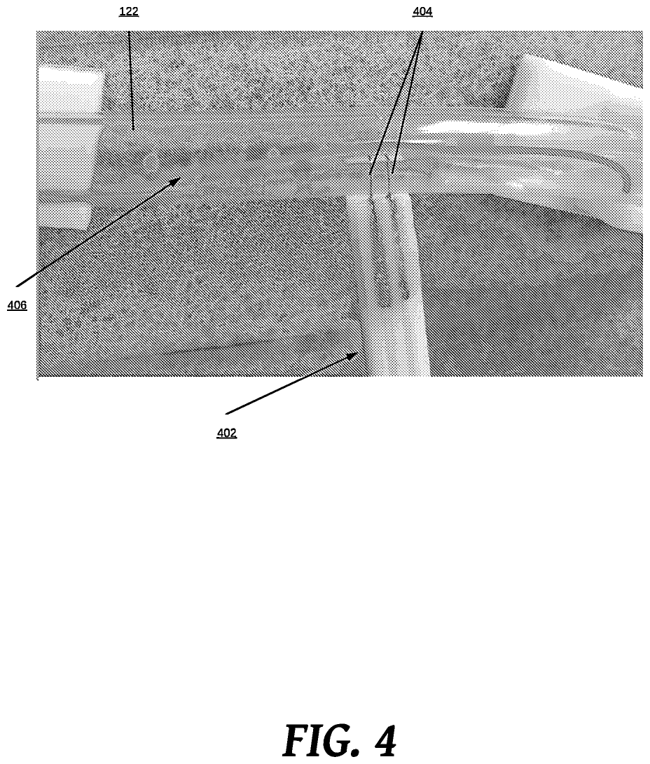

FIG. 4 illustrates an exemplary sensor 402 for measuring properties of urine. In various embodiments the sensor 402 may be a conductivity sensor including two or more electrodes 404 (e.g., 2, 3, 4, 5, 6, or more). The conductivity sensor 402 may measure the conductivity of urine inside the catheter tube 122 by passing a current through the urine sample via the electrodes 404. The current may be generated by applying a voltage, for example, an alternating voltage (AC) to one of the two or more electrodes 404 using a conductivity circuit. For example, an EZO EC conductivity circuit manufactured by Atlas Scientific. Therefore, the surface of the electrodes undergo reduction and oxidation to pass the current through to solution.

The current passed through the urine, may be used to measure the electrical resistance of the urine. In various embodiments, the conductivity circuit measures resistance of the two or more electrodes 404 based on the electrode's 404 predetermined cell constant (K). An electrode's 404 K constant may be calculated as a ratio of the distance the separating the electrodes 404 and the total surface area of the electrodes 404 (i.e., K=distance (L)/area (A)). To get conductivity of the urine the conductivity circuit takes the inverse of the resistance in ohms (Ω) to generate conductivity in Siemens per centimeter. Conductivity measurements may depend on the ionic characteristics of the urine. For example, the concentration and composition of the ions included in the urine. If there are no ions in the urine to carry charge between the electrodes no current will flow and the measured conductivity would be zero Siemens/cm. On the other hand, the more ions (e.g., sodium, potassium, chloride, creatinine, and the like) dissolved in the urine, the stronger the current flowing between the two or more electrodes 404 and the greater the conductivity. Therefore, conductivity data may be used to monitor kidney function by serving as a proxy for dissolved ion concentration. In various embodiments, the conductivity data may also be converted into a dissolved ion concentration to facilitate analysis of kidney function.

In various embodiments, each electrode 404 may comprise a conductive material such as copper, graphite, stainless steel, gold, and the like. The conductive material used for the electrodes may be durable and strong so that it can pierce the catheter tube 122 three or more times without deforming. The conductive material may also be corrosion resistant. The electrodes 404 may be configured to measure conductivity within the catheter tube 122 by penetrating into the catheter tube so that a portion of each electrode 404 is submerged in the liquid that passes through the catheter tube 122. To ensure accurate and consistent conductivity readings the portions of each electrode 404 inside the catheter tube should be fully submerged (i.e. below the liquid boundary line 406) in the urine contained in the catheter tube 122. Once the electrodes have penetrated into the catheter tube, the sensor 402 must secure each electrode 404 in place to maintain a consistent distance between each electrode. In various embodiments, changing the distance between the two electrodes 404 may disturb the accuracy of the conductivity data and/or require recalibration of the conductivity circuit.

Conductivity measurements may depend on the temperature of the urine. Therefore, in various embodiments, the conductivity circuit may be in communication with a temperature circuit to adjust the conductivity measurements to account for urine temperature. In various embodiments, the temperature circuit may measure temperature of the urine using a resistance temperature detector (RTD). For example, a OMEGA HYP4 hypodermic needle RTD controlled by a EZO RTD circuit manufactured by Atlas Scientific. In various embodiments, the temperature sensor may be inserted into the catheter tube adjacent to the electrodes 404 of the conductivity sensor. The temperature sensor may measure the temperature of the urine passing through the catheter tube by sending a voltage across a resistor located at the tip of the portion of the temperature sensor in contact with the liquid. Changes in the temperature of the resistor result in changes in the resistance of the resister. Resistance measurements made by the temperature sensor may then be interpolated by the temperature circuit based on prior calibrations that relate changes in temperature to changes in resistance. The calculated resistance compensation value for the measured temperature may then be sent to the conductivity control circuit before each conductivity measurement is made. Receiving the resistance compensation value in advance of measuring conductivity may allow the conductivity circuit to accurately account for urine temperature during each conductivity measurement.

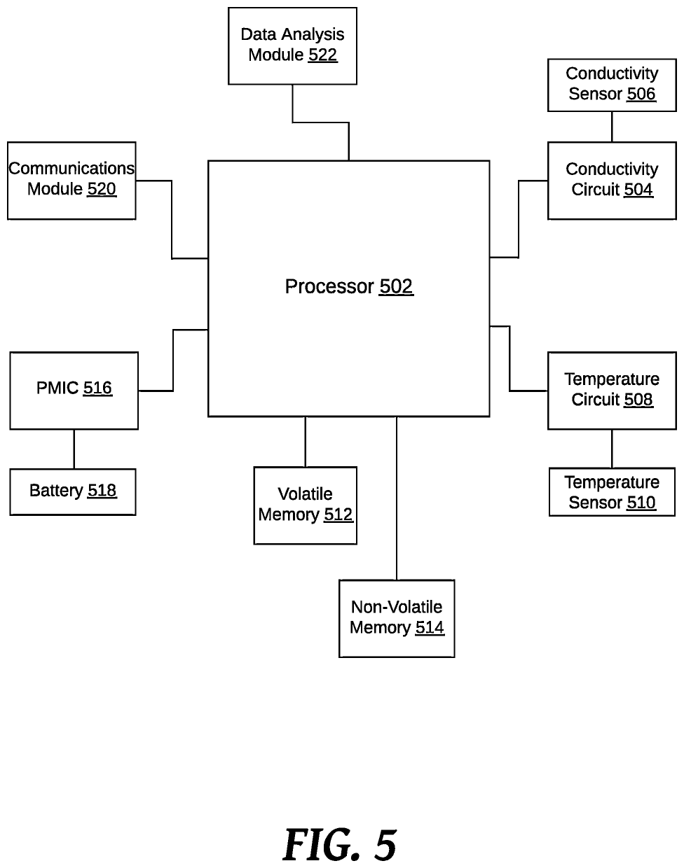

FIG. 5 illustrates a circuit diagram of an exemplary urine analysis device. As shown, the urine analysis device may include a printed circuit board (PCB) including various electronics components. In various embodiments, the PCB may include a processor 502 for executing commands and instructions of one or more of the components. Suitable processors 502 for the execution of a program of instructions may include, by way of example, both general and special purpose microcontrollers or microprocessors, and the sole processor or one of multiple processors or cores, of any kind of computer. In various embodiments, the processor 502 may be a ESP-8266 Feather microcontroller manufactured by Adafruit. Generally, a processor 502 may receive instructions and data from a volatile memory 512 or a non-volatile memory 514 or both. Suitable volatile memory 512 may include RAM, high speed memory, double data rate memory, 4R memory, and the like. Suitable non-volatile memory 514 may include embedded MMC or eMMC, solid-state drive or SSD, and the like.

The processor 502 may be coupled to one or more control circuits to control sensor input devices used to measure one or more properties of urine. In various embodiments, the urine analysis device measures conductivity of the urine using a conductivity sensor 506 controlled by a conductivity circuit 504. The conductivity sensor 506 may include one or more electrodes for measuring conductivity. The conductivity control circuit 504 may control one or more aspects of the conductivity sensor 506, for example, the sample rate of the conductivity sensor 506, the amount of voltage applied to the electrodes, and the like. The conductivity circuit 504 may include logic for ensuring a high sampling rate for conductivity measurements. For example, sampling rates of 1-5 seconds may be used to detect rapid changes in the conductivity of the liquid. The processor 502 may also facilitate communications between the conductivity circuit 504 and a temperature circuit 508 operating a temperature sensor 510 to automatically compensate for urine temperature in the conductivity measurements generated by the conductivity circuit. To improve the accuracy and reliability of conductivity data, the conductivity control circuit 504 may also include logic that controls transmission of conductivity samples to the communications module 520 and/or data analysis module 522. For example, the conductivity circuit 504 may ensure conductivity measurements are uploaded only when the electrodes are submerged in urine and or when the urine sample is stable and not flowing down the catheter tube.

Conductivity data generated by the conductivity circuit 504 may be transmitted to the data analysis module 522 for further analysis. In various embodiments, the data analysis module 522 may execute one or more operations to refine data conductivity data before it is transmitted to a remote computer device. For example, the data analysis module 522 may time stamp conductivity data points, average a series of conductivity measurements, convert the conductivity data to another form or unit, for example, dissolved ion concentration data, and/or perform other statistical operations to remove outliners, improve data quality, and/or facilitate analysis of conductivity data. The data analysis module may also generate one or more graphs, plots, charts, and/or other data visualizations that may be displayed on a remote device and/or a display integrated into the urine analysis device.

The processor 502 may interface with a communications module 520 to facilitate communicating with external devices. For example, the communication module 520 can include a wireless communications module for connecting to an external device (e.g., a laptop, an external hard drive, a tablet, a smart phone) for transmitting the data and/or messages to the external device. In various embodiments, the wireless communications module may include a Wi-Fi chip, an embedded Bluetooth module, and the like. The communications module 520 may transmit data using any known wired or wireless communications protocol, for example, Bluetooth, Wi-Fi, and the like. To facilitate faster communications, the communications module 520 may have multiple pins for connecting multiple parts of the conductivity circuit 504, temperature circuit 508, and/or data analysis module 522. A power management integrated circuit (PMIC) 516 may be integrated into the PCB and is responsible for controlling a battery charging circuit to charge a battery 518 or other power source. In various embodiments, the PCB may include built-in LiPoly charger that interfaces with a USB controller to charge the battery 518 by plugging a wall charger into a USB port coupled to the processor 502. The battery 518 supplies electrical energy for running the electrical components of the urine analysis device.

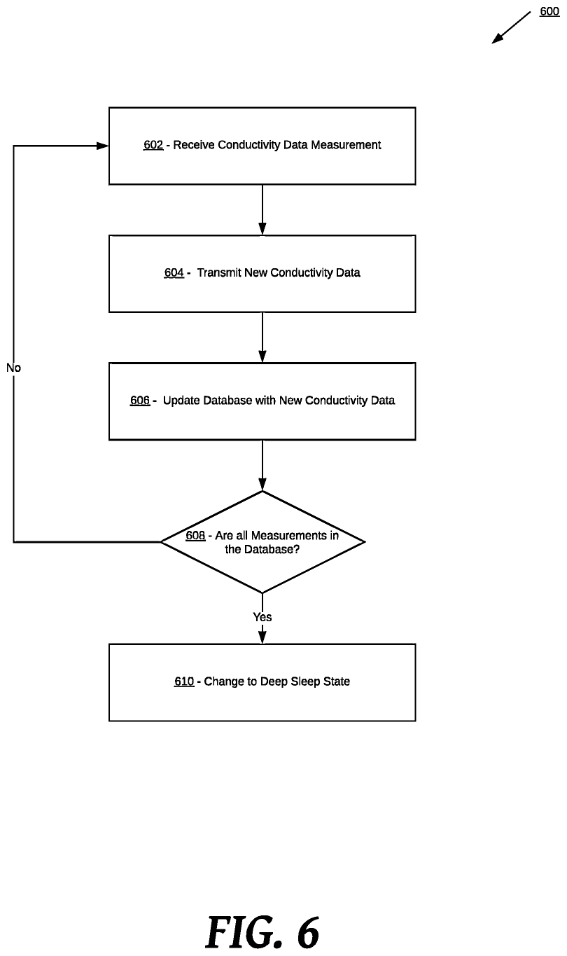

FIG. 6 illustrates an exemplary method for continuously recording conductivity measurements 600 using the urine analysis device. At 602, the processor may receive conductivity data measurements from a conductivity circuit. The processor may then execute one or more data transfer protocols of the communications module to transfer conductivity data measurements to a remote computer device at 604. In various embodiments, the communications module may include instructions for transferring conductivity data using a Wi-Fi communications protocol. This configuration allows the Wi-Fi chip and other aspects of the communication module specific to data transmission to be selectively powered off between data transmissions, for example, when no urine is being collected by the catheter system, between uploads of the new conductivity data, and the like.

At 606, new conductivity data collected by the conductivity circuit is added to a database. In various embodiments, the database is a cloud database accessible using one or more APIs. A webhook service may facilitate automatically updating the database with new conductivity data by establishing a connection between the communications module of the urine analysis device and a database linked to the communications module. To enable real time updates to the database, the webhook service may be integrated with the communication module to automatically establish a connection with the database whenever the communication module establishes a Wi-Fi connection.

In various embodiments, to update a database with new conductivity data using the webhook service, new conductivity data is measured by the conductivity circuit. Measuring new conductivity data triggers the processor to wake the communications module from a deep sleep state and establish a Wi-Fi connection. The webhook service, detects the Wi-Fi connection made by the communications module and pings the database. Using a database API, the webhook service then facilitates transmission of the new conductivity data from the communications module to the database. In various embodiments, the database may be a cloud database. For example, the database may be a Google cloud database hosting a Google Drive instance that stores a Google Sheets workbook. To communicate with the cloud database, the webhook service may be configured to connect to a cloud database API that interfaces with the cloud database. To automatically update the cloud database whenever new conductivity data is measured, the webhook service may be configured to automatically establish a connection with the cloud database API when the wireless communications device receives conductivity data from the conductivity circuit or other sensor data from the one or more sensors. The webhook service may then transmit the conductivity data to the cloud database via the cloud database API to update one or more records in the cloud database. In one or more embodiments, the cloud database may be included in a patient medical records system. The webhook service may connect to the patient medical records system using the cloud database API and transmit conductivity data to the patient medical records system via the cloud database API to provide real time updates of conductivity measurements included in patient medical records stored in the patient medical records system.

At 608, the communications module may determine if all of the conductivity data has been uploaded to the database. If the database is up to date, the communications module may change to a deep sleep state or other low power state at 610. For example, the communications module may change to a deep sleep state or other low power state by powering down the Wi-Fi chip and/or other aspects of the communications module to conserve battery power. In various embodiments, the communications module may automatically power down the Wi-Fi chip to a low power state upon receiving no new sensor data for one or more sampling periods of the conductivity circuit or one or more other sensors. If the database is not up to date, 602-606 may be repeated to update the database with new conductivity data.

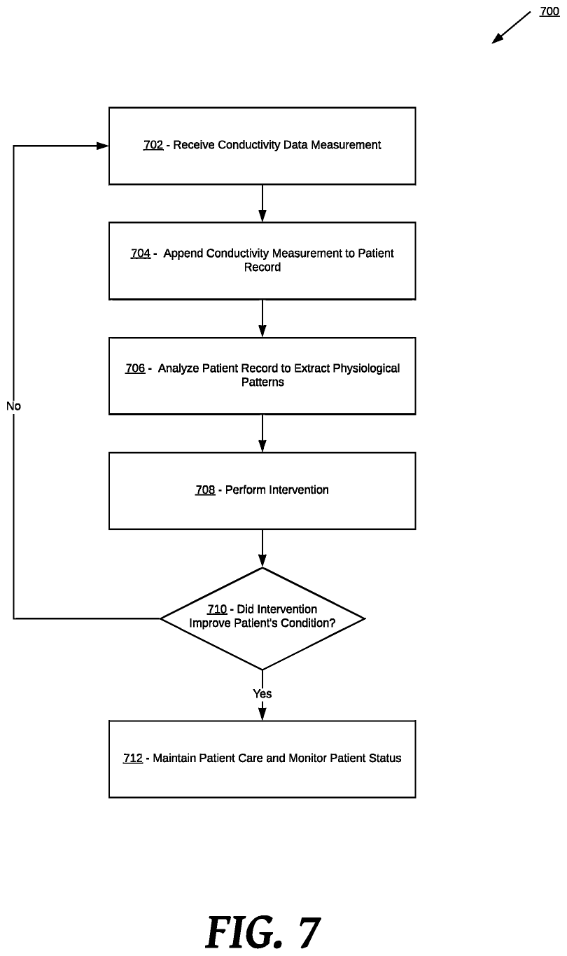

FIG. 7 is a flow chart illustrating a method of using the urine analysis device to monitor kidney function 700. At 702, conductivity data is received from a conductivity circuit. Using a communication module and/or a webhook service, the conductivity data may be appended to a patient record (i.e., a patient medical record) at 704. In various embodiments, the communication module and/or webhook service may be integrated with a database server to automatically upload new conductivity data in real time. For example, the webhook service may transmit the conductivity data to the database server to automatically update the patient record to include the conductivity data. The webhook service may automatically update the patient record to enable providers to view the patient's urine analysis results in real time. In various embodiments, the communications module and/or webhook service may be integrated with a medical records system to provide continuous and or real time updates to conductivity data persisted in patient medical records. For example, the communications module and or webhook service may include a wireless communications module that integrates with a cloud database API that interfaces with a patent medical records system. The wireless communications module may call the cloud database API to transmit conductivity data collected by the one or more sensors of the urine analysis system to the patient medical records system. To identify the patient that corresponds to the conductivity data, the communications module may attach a patient identification number, measurement time stamp, urine analysis system serial number and or other identification information for the patient and or the urine analysis system generating the conductivity data. The cloud database API may include programming logic for reading the conductivity data and any attached metadata, locating the medical record of the patient using the urine analysis system, and updating the patient's medical record in the patient medical records system with the new conductivity data.

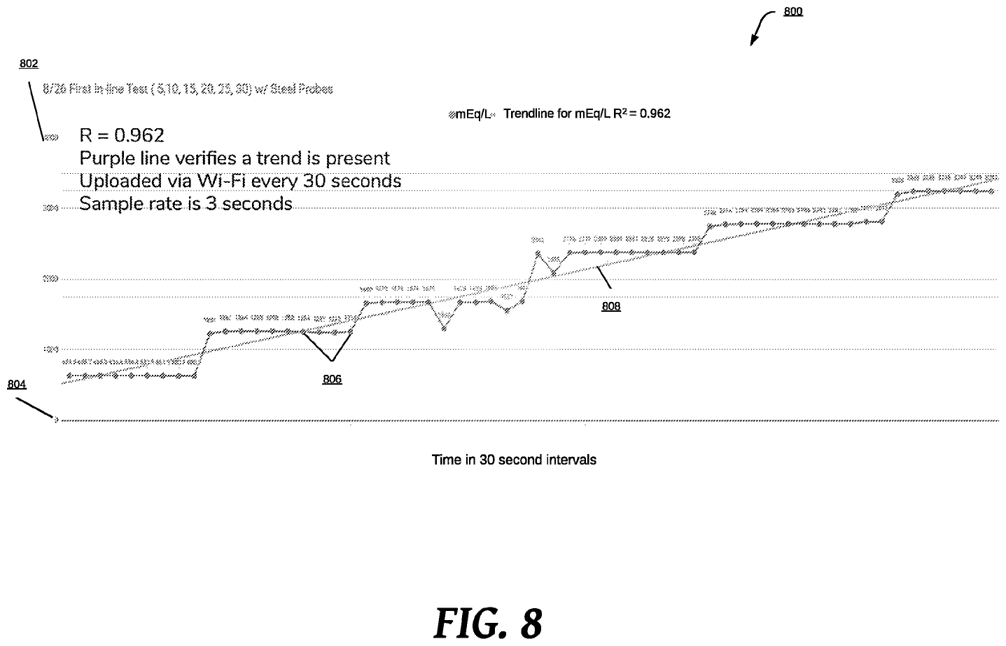

At 706, the conductivity data may be analyzed to extract one or more trends and/or patterns. For example, an increase and/or decrease in the conductivity of a urine sample. In various embodiments, the communications module and/or webhook service may also be integrated with a data analysis module to generate one or more graphs, plots, charts, and/or other data visualizations to facilitate analysis of conductivity data by one or more physicians and/or other health care service providers. FIG. 8 below illustrates one example of a graph that may be generated by a data analysis module to display conductivity data included in a patient record. To facilitate data analysis, the data analysis module may convert conductivity data into dissolved ion concentration, urine dissolved salts concentration, or other data format describing composition and/or volume of urine. In various embodiments, the data analysis module may automatically analyze the conductivity data to extract one or more tends and/or patterns and associate the extracted tends and/or patterns with changes in the patient's condition. The data analysis module may then send a notification physician or other healthcare service provider altering them of the extracted pattern and/or predicted changes in the patient's condition.

Trends in urine conductivity extracted from conductivity data may be used to monitor kidney function and other medical conditions. For example, low conductivity measurements may signal low concentrations of dissolved salts in urine and persistent low concentrations of dissolved salts over time may be interpreted as a sign of poor kidney function. Accordingly, a decrease in the patient's urine conductivity over time may be a trend extracted from the conductivity data that is used to determine the patient's kidney function is compromised. The one or more trends may be used to diagnose a patient condition. For example, a trend of a low and or decreasing urine conductivity may be associated with kidneys in a low protrusion state and/or acute kidney injury (AM). The conductivity data may be used as evidence to diagnose one or more conditions causing these symptoms.

Trends in urine conductivity may be interpreted in light of the clinical context and/or the individual patient receiving treatment. In various embodiments, trends in conductivity data may be used to differentiate the distinct causes of excessively high urine output. For example, if a patient with high urine output has a continuously elevated conductivity trend, this may indicate that the urine is very concentrated. In the proper clinical context, the elevated conductivity trend may suggest that the patient is actively wasting solutes and has salt-wasting syndrome, a condition that may follow a traumatic brain injury, brain tumors, intracerebral hemorrhage, and the like. In this case, the treatment is 3% saline. On the other hand, if a patient with high urine output has a continuously low conductivity trend, this suggests that the urine is diluted. In the proper clinical context, the decreased conductivity trend may indicate that the patient is actively wasting water and may be suffering from a form of Diabetes Insipidus (i.e., either central or nephrogenic). In this case, the possible intervention is to restrict the patient's fluid intake and/or initiate antidiuretic hormone replacement therapy.

Other treatments for acute kidney injury include, for example treatment with intravenous (IV) fluids, administration of diuretics, administration of medications to control blood potassium (e.g., sodium polystyrene sulfonate (Kionex)), medications to restore blood calcium levels, administration of calcium (e.g., calcium chloride, calcium gluconate, and calcium carbonate), dialysis, or combinations thereof. All treatments described herein can be combined with any other treatment.

In various embodiments, trends extracted from conductivity data may be used to monitor patient response to receiving a diuretic therapy. Observing an increased urine conductivity trend following a diuretic therapy indicates the diuretic is effective. On the other hand, a decreased urine conductivity trend, no discernable conductivity trend, and/or no change in the conductivity trend may indicate the diuretic therapy is not effective. In various embodiments, real time conductivity data may be used to monitor a patient response allowing the dose of the diuretic to be increased [within the safety range] until the trend starts to increase. Without real time urine conductivity data, there is no way to rapidly tell the effect of a particular dose of diuretic and diuretic therapies are administered without reliable data to measure their effect.

Trends in urine conductivity may also be used to monitor a patient's hydration status. If a patient is dehydrated, the kidney starts to conserve water and the urine conductivity will increase resulting in an increasing conductivity trend. Conversely, if a patent is hydrated the urine conductivity will be relatively low and a decreasing conductivity trend may be detected as a patient transitions from a dehydrated to hydrated state. Patient hydration is important to monitor in particular clinical contexts. For example, scenarios in which a patient needs a radiology test that uses an IV contrast dye. This type of dye may injure the kidney in a patient that is dehydrated. Therefore, a physician detecting a high conductivity trend in prior to the administration of contrast dye may give the patient IV fluids to correct the dehydration. Once the conductivity trend decreases indicating that the kidney does not need to conserve extra water, the contrast dye may be administered and the patient can safely go to her radiology test. Even in situations where the patient is not dehydrated, administering IV fluids until a decrease in the conductivity trend is observed in the patient's urine before injecting the contrast dye may help mitigate any possible kidney injury caused by the dye.

At 708, a physician may perform an intervention based on the trends extracted from the urine conductivity data and the clinical context. For example, upon observing a trend of low urine conductivity, the physician may, depending on the clinical context, give the patient more blood to increase blood flow to the patient's kidneys and/or other organs, restrict the patients fluid intake, initiate antidiuretic hormone replacement therapy, increase a diuretic dose, or perform another intervention to increase the patient's urine conductivity trend. After observing a trend of high urine conductivity, the physician may, depending on the clinical context, administer IV solutions (e.g., 3% saline solution and the like) or perform another intervention to decrease the patient's urine conductivity trend.

Conductivity data may be continuously monitored after performing the patient intervention to monitor the patient's response to the intervention. At 710, conductivity data collected after the intervention may be analyzed to determine if the patient's condition improved. If the patient's condition did improve after the intervention, the course of care may be maintained and the patient's status may be monitored at 712 until the patient fully recovers. If the patient's condition did not improve after the intervention, the steps 702 to 708 may be repeated to determine the patient's current kidney function and the same and/or different intervention may be performed to attempt to improve the patient's condition.

FIG. 8 illustrates an exemplary graph of conductivity data 800 that may be generated using conductivity data detected by conductivity control sensor. As shown, the graph 800 may show conductivity data for a patient over time. In various embodiments, conductivity data may be shown one the vertical axis 802 and time may be shown on the horizontal axis 804. In FIG. 8, the horizontal axis, for example, shows a sample taken every 30 seconds. The conductivity data may be in given as a raw voltage value and/or converted to another unit, for example, Siemens per meter (S/m). The data analytics module may also convert conductivity data into a measure of concentration, for example, milliequivalent per liter (mEq/L), to facilitate analysis of the urine ion level.

In various embodiments, conductivity data may be sampled every 1, 2, 3, 4, 5, 10, 30, 45, or 60 seconds or 2, 3, 4, 5, 10, 20 minutes or more. These conductivity data samples, for example, every 3 seconds may be averaged to generate one conductivity measure 806 every 30 seconds (or any other suitable amount of time, e.g., about every 10, 20, 30, 40, 50, or 60 seconds, or every 2, 5, 10, 20, 30, 40, 50, or 60 minutes, or any range between about 10 seconds and 60 minutes). For example, the ten conductivity data samples collected during a 30 second period may be averaged to generate one conductivity measure 806 for the 30 second period. The conductivity measures 806 are then uploaded a database and plotted on a graph by the data analytics module. In various embodiments, averaging the conductivity data samples reduces the amount of noise in the data attributable to variation in the amount of liquid in the catheter tube, flow rate during a particular measurement, temperature of the urine, and the like. A data analysis module may then analyze the conductivity data by drawing one or more trendlines 808. For example, trendlines 808 may be drawn to indicate and increase and/or decrease in the urine conductivity and/or ion level.

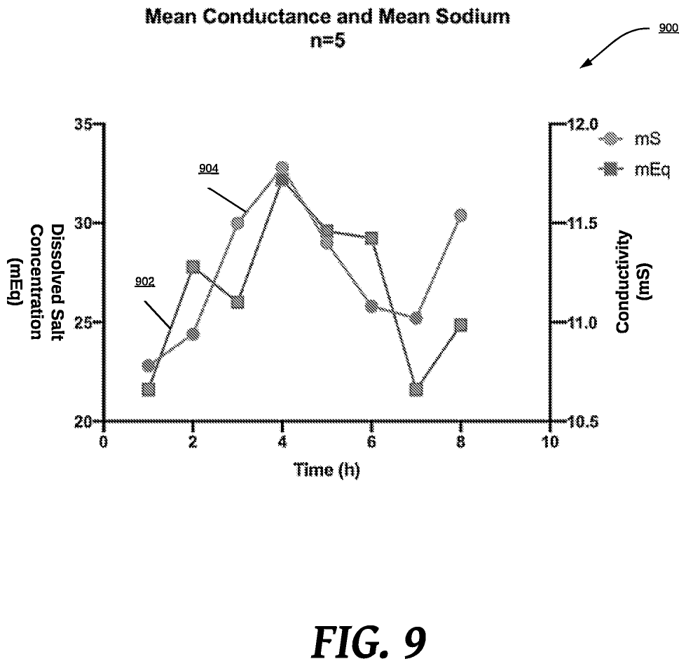

FIG. 9 is a line chart 900 that illustrates the relationship between urine conductivity and dissolved salt concentration. As described above, the urine analysis system measures urine conductivity. Urine conductivity is closely related to the patient's dissolved salt concentration, therefore, urine conductivity data may be used to track the patient's hydration level. The line chart 900 illustrates urine conductivity and dissolved salt concentration for 5 patients measured over an 8 hour period. Measurements for each patient were taken every hour and the measurements for all 5 patients were averaged to get the conductivity values and the dissolved salt concentration values shown in the line chart 900. All of the 5 patients included in the dataset were in a state of shock therefore each patient's condition was stable during the 8 hour measurement time period. The values for the average dissolved salt concentration (i.e., the mean values) are shown in the line chart 900 as rectangles connected by a first line 902. All dissolved salt concentration values are in milliequivalents (mEq) and the range of mean values for dissolved salt concentration ranges from a high of 32.5 mEq at hour 4 to a low of 21.5 mEq at hour 1. The values for the average conductivity (i.e., the mean values) are shown in the line chart 900 as circles connected by a second line 904. All conductivity values are in millisiemens (mS) and the range of mean values for conductivity ranges from a high of 11.7 mS at hour 4 to a low of 10.6 mS at hour 1.

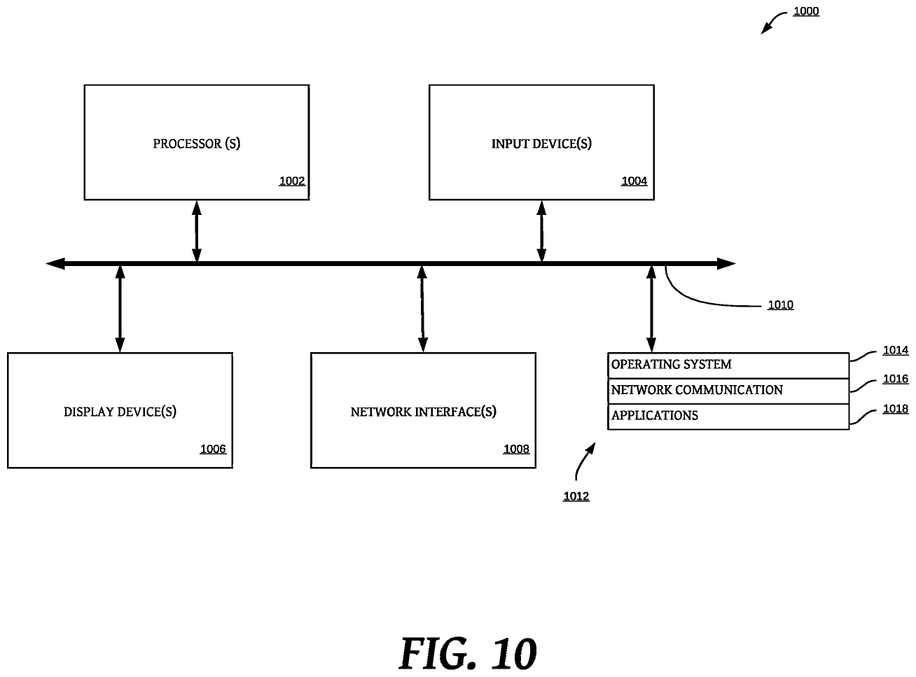

As shown in the line chart 900, the dissolved salt concentration largely changes with the conductivity. This relationship is shown by the changes in direction of the second line 904 largely mimicking the changes in direction of the first line 902. For example, when the second line 904 rises (i.e., conductivity increases), the first line 902 also rises (i.e., dissolved slat concentration increases) and when the second line 904 falls (i.e., conductivity decreases), the first line 902 also falls (i.e., dissolved slat concentration decreases). Therefore, using urine conductivity as a proxy for dissolved salt concentration is supported by the experimental data shown in the line chart 900 in FIG. 9. FIG. 10 shows a computing device according to an embodiment of the present disclosure. For example, computing device 1000 may function as client 160 (which may include a system for analyzing and/or displaying urine data). The computing device 1000 may be implemented on any electronic device that runs software applications derived from compiled instructions, including without limitation personal computers, servers, smart phones, media players, electronic tablets, game consoles, email devices, etc. In some implementations, the computing device 1000 may include one or more processors 1002, one or more input devices 1004, one or more display devices 1006, one or more network interfaces 1008, and one or more computer-readable mediums 1012. Each of these components may be coupled by bus 1010, and in some embodiments, these components may be distributed among multiple physical locations and coupled by a network.

Display device 1006 may be any known display technology, including but not limited to display devices using Liquid Crystal Display (LCD) or Light Emitting Diode (LED) technology. Processor(s) 1002 may use any known processor technology, including but not limited to graphics processors and multi-core processors. Input device 1004 may be any known input device technology, including but not limited to a keyboard (including a virtual keyboard), mouse, track ball, camera, and touch-sensitive pad or display. Bus 1010 may be any known internal or external bus technology, including but not limited to ISA, EISA, PCI, PCI Express, NuBus, USB, Serial ATA or FireWire. Computer-readable medium 1012 may be any medium that participates in providing instructions to processor(s) 1002 for execution, including without limitation, non-volatile storage media (e.g., optical disks, magnetic disks, flash drives, etc.), or volatile media (e.g., SDRAM, ROM, etc.).

Computer-readable medium 1012 may include various instructions 1014 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 1004; sending output to display device 1006; keeping track of files and directories on computer-readable medium 1012; controlling peripheral devices (e.g., disk drives, printers, etc.) which can be controlled directly or through an I/O controller; and managing traffic on bus 1010. Network communications instructions 1016 may establish and maintain network connections (e.g., software for implementing communication protocols, such as TCP/IP, HTTP, Ethernet, telephony, etc.).

Application(s) 1018 may be an application that uses or implements the processes described herein and/or other processes. For example, a data analysis application that generates conductivity data visualizations, analyzes conductivity data to generate diagnostic predictions, executes one or more operations to transform conductivity data to another unit, facilitates manual analysis by physicians, and the like. The processes may also be implemented in operating system 1014. For example, application 1018 and/or operating system 1014 may present UIs 162 including urine data 164 which may include results from data analysis tasks as described herein.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, microcontrollers, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings.

Any single term, single element, single phrase, group of terms, group of phrases, or group of elements described herein can each be specifically excluded from the claims.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A system for urine analysis integrated with a catheter, the system comprising:
a clamp connected to a catheter tube and a body portion mounted to a urine collection receptacle, the body portion comprising a sensor housing disposing one or more sensors configured to collect sensor data describing one or more properties of urine collected by the catheter;
a communications module configured to update a patient record by transmitting the sensor data to a database; and
a data analytics module configured to generate one or more data visualizations displaying the sensor data.

2. The system of claim 1, wherein the one or more sensors include a conductivity sensor configured to collect conductivity data describing a dissolved ion concentration of urine.

3. The system of claim 1, further comprising the communications module including a wireless communications module configured to continuously update the patient record in real time by integrating with a cloud database API that interfaces with a patent medical records system.

4. The system of claim 3, wherein the wireless communications device is a Wi-Fi chip integrated with a webhook service.

5. The system of claim 4, further comprising, the webhook service configured to automatically establish a connection with the cloud database API when the wireless communications device receives sensor data.

6. The system of claim 4, further comprising, upon receiving no new sensor data for one or more sampling periods, the communications module is configured to transition the wireless communications device to a low power state having the Wi-Fi chip powered off

7. The system of claim 2, wherein the one or more visualizations include a graph displaying patient conductivity data collected over a period of time.

8. The system of claim 7, further comprising the data analytics module configured to extract one or more trends from the graph by drawing a trendline indicating an increase or decrease in urine conductivity.

9. A urine salt monitoring device integrated with a catheter system, the device comprising:
a clamp connected to a catheter tube included in the catheter system;
a body portion mounted to a urine collection receptacle included in the catheter system;
a sensor housing disposed in a cavity included in the body portion, the sensor housing disposing one or more sensors configured to collect sensor data describing one or more properties of urine collected by the catheter; and
a processor and memory disposed inside the body portion, the memory including instructions causing the processor to:
operate the one or more sensors to generate the sensor data; and
continuously upload the sensor data to a database to update a patient record in real time.

10. The device of claim 9, wherein the one or more sensors include a conductivity sensor configured to measure the conductivity of urine collected by the catheter system.

11. The device of claim 10, wherein the conductivity sensor includes two or more electrodes configured to be inserted into the catheter tube to contact urine as it flows through the catheter tube.

12. The device of claim 11, further comprising a cap placed over the sensor housing, the cap having a groove for disposing the catheter tube and one or more openings within the groove.

13. The device of claim 12, wherein the two or more electrodes extend through the two or more openings to contact the catheter tube disposed inside the groove.

14. The device of claim 13, further comprising the clamp configured to rotate over the groove to compress the catheter tube against the two or more electrodes, the clamp dimensioned to force the electrodes to pierce the catheter tube when the clamp is secured over the groove.

15. The device of claim 9, further comprising a top clip and a bottom clip for shaping the catheter tube into an elbow bend that forces a portion of the catheter tube extending over a top surface of the body portion to lay horizontally.

16. The device of claim 15, wherein the elbow bend increases dwell time of urine in a portion of the catheter tube disposed inside the clamp.

17. A method of using a system for urine analysis integrated with a catheter to determine kidney function, the method comprising,
measuring conductivity data of urine collected by the catheter;
updating a patient record to include the conductivity data;
analyzing the patient record to extract one or more trends from the measured conductivity data; and
determining kidney function of a patient based on the one or more trends.

18. The method of claim 17, wherein the one or more trends comprise a decrease in the patient's urine conductivity over time and the patient's kidney function is determined to be compromised based on the decrease in the patient's urine conductivity.

19. The method of claim 17, further comprising converting the measured conductivity data to a urine dissolved salts concentration.

20. The method of claim 17, further comprising performing an intervention on the patient to improve the patient's kidney function.
